(19) United States
(12) Patent Application Publication
(10) Pub. No.: US 2005/0095264 A1
(43) Pub. Date: May 5, 2005

(54) Volatile material-containing compositions having a consistent release profile

(75) Inventors: Fernando Ray Tollens, Cincinnati, OH (US); Jonathan Robert Cetti, Mason, OH (US); Michael Rene Weaver, Cincinnati, OH (US); Glenn Thomas Jordan IV, Indian Springs, OH (US); Christaain Arthur Jacques Kamiel Thoen, West Chester, OH (US); Zaiyou Liu, West Chester, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/921,670

(22) Filed: Aug. 19, 2004

Related U.S. Application Data
(63) Continuation-in-part of application No. 10/447,749, filed on May 29, 2003.
(60) Provisional application No. 60/587,405, filed on Jul. 13, 2004.

(51) Int. Cl. A61K 7/32; A61K 35/78

(52) U.S. Cl. 424/401; 424/725

(57) ABSTRACT

Volatile material-containing compositions having a consistent release profile and methods of emitting volatile materials from a volatile material-containing composition in a consistent manner are disclosed.

### CROSS-REFERENCE TO RELATED APPLICATIONS

This application is a continuation-in-part to U.S. application Ser. No. 10/447,749 filed May 29, 2003 and to U.S. Provisional Application Ser. No. 60/587,405 filed Jul. 13, 2004.

### FIELD OF THE INVENTION

The present invention relates to volatile material-containing compositions having a consistent release profile and methods of emitting volatile materials from a volatile material-containing composition in a consistent manner.

### BACKGROUND OF THE INVENTION

Volatile material-containing compositions are used for various purposes. Such purposes include, but are not limited to releasing into a room or other space, volatile materials such as perfumes or scented materials, insecticides, air fresheners, deodorants, aromacology, aromatherapy, or any other volatile that acts to condition, modify, or otherwise charge the atmosphere or to modify the environment.

There are several drawbacks to known compositions used for these purposes. One significant drawback is the fact that, over time, the character of the volatile materials being released may change. In the case of a volatile having multiple perfume ingredients, this results in a change in the overall scent. Commonly, the more highly volatile perfume ingredients (referred to as the “top notes” and the “middle notes”) are depleted before the less volatile bottom notes. This results in a confusing situation for the user, since the device still emits a scent, but the scent character is different (predominately “bottom notes”). Unfortunately, a predominately “bottom note” scent is usually not as desirable as the full perfume profile. It would be preferable to have all the “notes” emitted in the same relative percentages, thereby producing the same scent, throughout the useful life of the product. Therefore, a need still exists for a means to provide a consistent release profile of the volatile components throughout the useful life of a volatile-containing material.

### SUMMARY OF THE INVENTION

This invention relates to volatile material-containing compositions having a consistent release profile and methods of emitting volatile materials from a volatile material-containing composition in a consistent manner. Several non-limiting embodiments are described herein, each of which may constitute an invention in its own right or together with other components. In one non-limiting embodiment, the volatile material-containing composition comprises a carrier, at least one volatile material having at least a first component and a second component, where the first component and the second component have different evaporation rates; and at least one polymer having a Hydrophobicity Index greater than about 1.0 and lower than about 3.0. The composition has a first state when energy is not applied to the composition, and a second energized state when energy is applied to the composition. The volatile material is emitted at a first level from the volatile material-containing composition in the first state and the volatile material is emitted from the volatile material-containing composition at a second higher level in the second state. The volatile material-containing composition returns to the first state when energy is no longer applied to the volatile material-containing composition. Preferably, the composition releases less than 10 mg/hour of the volatile material at 25° C. and 50% relative humidity (RH).

Methods of releasing a volatile material into the atmosphere while providing a consistent odor profile of the volatile material are also disclosed.

Numerous other embodiments are also possible, including, but not limited to those described in the following detailed description.

### DETAILED DESCRIPTION OF THE INVENTION

This invention relates to volatile material-containing compositions having a consistent release profile and methods of emitting volatile materials from a volatile material-containing composition in a consistent manner. Several non-limiting embodiments are described herein, as are several components of the system, each of which may constitute an invention in its own right or together with other components.

The volatile materials can be emitted in various facilities, which include but are not limited to rooms, houses, hospitals, offices, theaters, buildings, and the like, or into various vehicles such as trains, subways, automobiles, airplanes and the like.

The term “volatile materials” as used herein, refers to a material that is vaporizable. The terms “volatile materials”, “aroma”, and “scents”, as used herein, include, but are not limited to pleasant or savory smells, and, thus, also encompass scents that function as insecticides, air fresheners, deodorants, aromacology, aromatherapy, or any other volatile that acts to condition, modify, or otherwise charge the atmosphere or to modify the environment. It should be understood, however, that perfumes, aromatic materials, and scents will often be comprised of one or more volatile materials (which may form a unique and/or discrete unit comprised of a collection of volatile materials).

The term “carrier,” as used herein, refers to a material that is a solid at room temperature and is the primary component in addition to the volatile material. Preferably, the carrier is a pliable solid at room temperature. Useful carriers include polyethylene glycol, hydrogenated castor oil and high chain fatty acids, particularly those with a chain length of greater than or equal to 14 carbon atoms.

The term “release profile,” as used herein, refers to the relative evaporation rate of individual volatile components within a mixture of volatile components. These volatile components have different volatilities, boiling points, and odor detection thresholds. When a volatile composition is discharged into the air, the ingredients with the higher volatilities (referred to as “top notes”) will be the ingredients that will volatilize and be detected by a person's sense of smell more quickly than the ingredients with lower volatilities (referred to as “middle notes”) and the ingredients with the lowest volatility (referred to as “bottom notes”). This will cause the character of the perfume to change over time since after the perfume is first emitted, the overall perfume character will contain fewer and fewer top notes and more bottom notes. The term “consistent release profile” is defined as a perceivable volatile component intensity and character that is comparable to its initial perfume intensity and character and that this is maintained for most of the intended use expectancy of the product. In other words, a composition where the proportions of top, middle and bottom notes stay relatively proportional during the intended usage. Preferably, the carrier of the present invention does not interfere with the release profile of the volatile material.

The term “Hydrophobicity Index,” as used herein; is determined as follows:

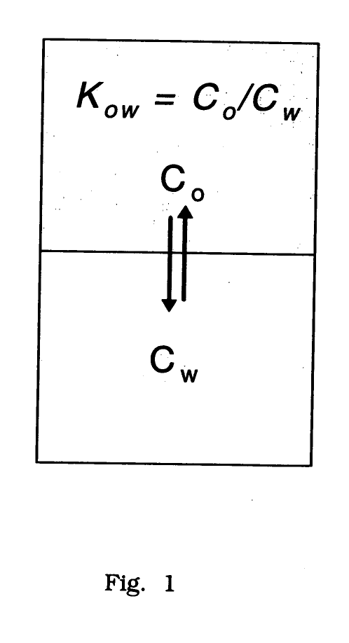

The hydrophobicity of a given molecule can be defined by its partitioning coefficient between organic and aqueous (water) phases (P_{ow}) A commonly used organic phase for such purpose is n-octanol. For convenience log (P_{ow}) (or cLog (P)) is often used to rank and compare the hydrophobicity of organic compounds, and perfume raw materials. Higher clog (P) values means higher hydrophobicity, and vise versa. (See FIG. 1)

FIG. 1. Illustration of Partitioning Between Organic and Aqueous Phases

A similar approach can be applied to polymeric molecules. These can be either simple polymers that are composed of repeating units of single monomer moieties, such as polyethylene; or co-polymers, which are composed of two or more structurally different repeating moieties.

The hydrophobicity of polymers is estimated using weight averaged clog (P) of individual repeating moieties in the polymer. The estimated hydrophobicity value for polymers in this manner is defined here as Hydrophobicity Index (PHI). As an example, the PHI of a polymer having three structurally different repeating monomer moieties (X,Y and Z) can be estimated as follow:
PHI=W_{X} (logP_{X})+W_{Y} (logP_{Y})+W_{Z} (logP_{Z}) (1)
where W_{X} is the weight percent of the monomer moiety X, W_{Y} is the weight percent of the monomer moiety Y, and W_{Z} is the weight percent of the monomer moiety Z. P_{X} is the partitioning coefficient of the monomer moiety X, P_{Y} is the partitioning coefficient of the monomer moiety Y, and P_{Z} is the partitioning coefficient of the monomer moiety Z. The partitioning coefficient of each of the monomer moiety is defined by K_{ow} (see FIG. 1 above). As cLog (P) defines hydrophobicity for simple molecules, PHI approximates the hydrophobicity of polymers.

Polymer Hydrophobicity Index (PHI) is a concept used to approximate the affinity of a polymer for simple molecules. Perfume partitioning into a polymer matrix can be qualitatively estimated using their cLog (P) and the PHI of the corresponding polymer. In general, higher affinity (partitioning) is expected for perfume ingredients with similar hydrophobicity values as that of the polymer itself.

While not wanting to be bound by theory, it is believed that polymers with a PHI in the range similar to the cLog (P) of perfume ingredients provide a higher degree of impact. Therefore, selecting polymers that have higher affinity for the more volatile portion of the perfume ingredients (i.e., perfume ingredients with KI values<1200), suppresses the evaporation rates of the perfume ingredients under heated conditions to give a slower decay rate over time. This provides benefits for delivering more consistent perfume presentations, thus odor character integrity, over a longer portion of the consumer usage period.

Kovat's Index (KI, or Retention Index) is defined by the selective retention of solutes or perfume raw materials (PRMs) onto the chromatographic columns. It is primarily determined by the column stationary phase and the properties of solutes or PRMs. For a given column system, a PRM's polarity, molecular weight, vapor pressure, boiling point and the stationary phase property determine the extent of retention. To systematically express the retention of analyte on a given GC column, a measure called Kovat's Index (or retention index) is defined. Kovat's Index (KI) places the volatility attributes of an analyte (or PRM) on a column in relation to the volatility characteristics of n-alkane series on that column. Typical columns used are DB-5 and DB-1.

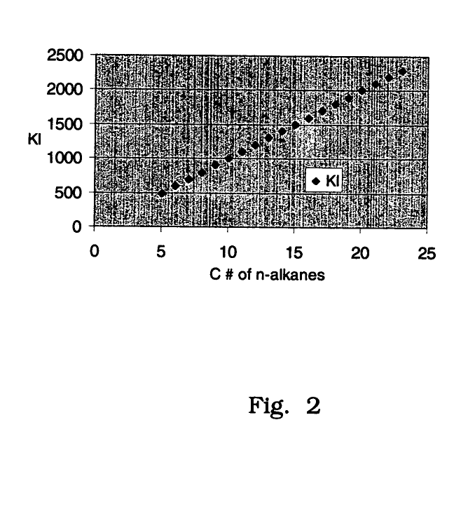

By this definition the KI of a normal alkane is set to 100n, where n=number of C atoms of the n-alkane. It can be shown that they are related in FIG. 2.

FIG. 2. Kovat's Index of n-alkanes on GC columns With this definition, the Kovat's index of a PRM, x, eluting at time t′, between two n-alkanes with number of carbon atoms n and N having corrected retention times t′ₙ and t′_{N} respectively will then be calculated as:

$$KI = 100 ( n + \frac{log {t}_{x}^{′} - log {t}_{n}^{′}}{log {t}_{N}^{′} - log {t}_{n}^{′}} ) \qquad ( 2 )$$

On a non-polar to slightly polar GC stationary phases, KI of PRMs are correlated with their relative volatility. For example, PRMs with smaller KI tends to be more volatile than that with larger KI. Ranking PRMs with their corresponding KI values give a good comparison of PRM evaporation rates in liquid-gas partitioning systems.

In a preferred embodiment, the polymer used in the present invention has a Hydrophobicity Index greater than about 1.0 and lower than about 3.0. More preferably, the polymer used in the present invention has a Hydrophobicity Index greater than about 1.0 and lower than about 2.5. Still more preferably, the a Hydrophobicity Index greater than about 1.0 and lower than about 2.0.

Preferred polymers include polystyrene, bimodal polystyrene, polybutadiene, poly(methyl methacrylate), polyurethane, blends of polyurethane and rosin plasticizer, and mixtures thereof. More preferably, the polymer is poly(methyl methacrylate) and polybutadiene.

In a preferred embodiment, the composition contains at least about 1% of polymer by weight. More preferably, the composition contains at least about 5% of polymer by weight. Still more preferably, the composition contains at least about 10% of polymer by weight.

In one non-limiting embodiment, the volatile material-containing composition comprises a carrier, at least one volatile material, and at least one polymer. The composition has a first state when energy is not applied to the composition, and a second energized state when energy is applied to the composition. The volatile material is emitted at a first level from the volatile material-containing composition in the first state and the volatile material is emitted from the volatile material-containing composition at a second higher level in the second state. The volatile material-containing composition returns to the first state when energy is no longer applied to the volatile material-containing composition.

Preferably, the composition releases less than 10 mg/hour of the volatile material at 25° C. and 50% relative humidity (RH). More preferably, the composition releases less than 5 mg/hour of the volatile material at 25° C. and 50% relative humidity (RH). Even more preferably, the composition releases less than 1 mg/hour of the volatile material at 25° C. and 50% relative humidity (RH).

In one embodiment, a system for dispensing scents into the environment can be provided which comprises one or more components containing one or more scents or aromatic materials. In such an embodiment, the system preferably comprises a dispensing device, such as a device and one or more aromatic material-containing articles of manufacture, or “scent-containing articles of manufacture”, which may be provided in the form of fragrance “cartridges”. Each cartridge can provide a single volatile composition, or a combination of different volatile materials, such as a combination of different scented materials. In certain embodiments, each of the cartridges provides a collection of scents that conveys, e.g., a theme, an experience, a physiological effect, and/or a therapeutic effect.

The volatile compositions of interest herein can be provided in any suitable form. In some embodiments, scents are provided by volatile compositions comprising perfume, such as perfume oils, that are incorporated onto or into a suitable carrier. The carriers can be provided in the following non-limiting forms: a solid, a liquid, a paste, a gel, beads, encapsulates, wicks, a carrier material, such as a porous material impregnated with or containing the perfume, and combinations thereof. In some embodiments, the carrier is in the form of a pliable solid which can be melted and have the perfume ingredients added thereto in order to form a composition that is in the form of a pliable solid structure or matrix at room temperature (73° F. (25° C.), 50% RH).

In certain embodiments, the volatile composition has a viscosity of from about 1,000 Cps to about 1,000,000 Cps, or more, measured at a shear stress of 100 Pa in a rotational rheometer, like the AR2000 (TA instruments New Castle, Delaware, USA), using a 40-mm diameter cone-and-plate geometry at 25 ° C. Such a composition can exist as a gel up to at least about 13,000 Cps. In certain embodiments when the composition is in the form of a pliable solid, it can have a viscosity of from about 100,000 to about 1,000,000 Cps.

In one non-limiting embodiment, at room temperature, the composition is in the form of a structure that is a structured polymeric pliable solid. The structure may be homogeneous (which may also be referred to herein as “continuous”), or non-homogeneous. In many embodiments, it is desirable for the structure to be permeable to volatile materials contained therein. This will allow the structure to release the volatile materials contained therein when desired. In preferred versions of such an embodiment, the composition comprises a non-porous, homogeneous, permeable, structured polymeric pliable solid.

The volatile composition can be formed in a number of different manners. In one embodiment, the composition can be made by adding the volatile ingredient(s) to a carrier, such as polyethylene glycol (or “PEG”). The volatile ingredients, such as perfumes, are preferably miscible with the carrier, and after cooling, forms a pliable solid-like at room temperature. PEG is available in various molecular weights. While PEG's having low molecular weights (or “MW”) (e.g., molecular weights less than 400) can be used as solvents for perfumes, such PEG's are liquids at room temperature, and may be used, but are not preferred for use in the compositions described herein. In more preferred embodiments of the composition, the MW of PEG is greater than or equal to about 1,000, or greater than or equal to about 4,000. It is desirable that the MW of PEG be greater than or equal to about 8,000. The molecular weight of PEG may be as high as 24,000, or higher. All molecular weights specified herein are weight average molecular weights.

Other suitable carriers are hydrogenated castor oil and high chain fatty acids, particularly those with a chain length of greater than or equal to 14 carbon atoms. In certain embodiments, it is desirable for the majority of the composition to comprise such a carrier and the volatile ingredient(s). Thus, such a carrier and the volatile ingredient(s) may comprise more than about 20%, alternatively, more than about 50% of the composition, by weight. In certain embodiments, it may be desirable for the composition (and/or the carrier) to also be substantially free of HPC (hydroxy propyl cellulose).

It may be desirable to utilize a structurant with the carrier. A structurant can be used for any suitable purpose. Examples of such purposes include, but are not limited to providing the structure formed by the composition with greater stability. The structurant can reduce the tendency of the structure to release the volatile material(s) at low temperatures (e.g., ambient or storage or shipping temperatures). Thus, the volatile material(s) will not be released until energy is applied to the structure in order to release the volatile material(s). Any suitable structurant can be used. Suitable structurants comprise any substance that includes a divalent cation. Substances that comprise divalent cations include, but are not limited to magnesium and calcium containing molecules such as magnesium and calcium chloride, magnesium and calcium carbonate. Other suitable structurants include, but are not limited to derivatives of castor oil, including, but not limited to hydrogenated castor oil.

It may also be desirable for the composition to include at least one wax. Waxes can be used for any suitable purpose, including, but not limited to raising the melting temperature of structure formed by the composition for improved stability. Any suitable wax(es) can be used. In certain embodiments, it is desirable for the wax to have a melting point that is greater than that of the carrier. If the carrier is PEG, the melting point of the wax may, for example, be greater than about 50° C. Suitable waxes include, but are not limited to waxes that are derivatives of the carrier, for example, derivatives of PEG. Waxes that are derivatives of the carrier may be preferred because the structurants that are capable of structuring the carrier will also be able to structure the waxes in order to further raise the melting point of the entire matrix. It may also be desirable that the wax does not have an affinity for the volatile material so that it does not affect the emission rate or delivery of the volatile material.

In one embodiment, the composition is formed by combining polyethylene glycol (or “PEG”), hydrogenated castor oil, and a low level of at least one wax, at least one volatile ingredient, and at least one polymer having a Hydrophobicity Index greater than about 1.0 and lower than about 3.0.

The volatile ingredient(s) can comprise a number of components or compositions, including, but not limited to: fragrances (or perfume oils), flavors, pesticides, repellants, or mixtures thereof.

The volatile ingredient(s) can be combined with the carrier material in any suitable manner. Several suitable manners in which the volatile ingredient(s) can be combined with the carrier material include, but are not limited to: by entrapment; the volatile ingredient(s) can be dissolved in the carrier material; the volatile ingredient(s) can be partially encapsulated or completely encapsulated in the carrier material.

The components of the composition can be incorporated into the composition in any suitable amounts. In some embodiments, it may be desirable for the concentration of the volatile material(s) to be greater than about 5% of the composition. More preferably, the concentration of the volatile material(s) is greater than about 10% of the composition. In some embodiments, the concentration of the volatile material(s), such as the perfume ingredients, may be as high as about 75%, or more of the composition. In other embodiments, the amount of volatile material(s) may range from about 25% to about 75% of the composition. The carrier (such as polyethylene glycol) may comprise the balance of the composition. In some embodiments, the carrier may range from about 25% to about 75%, or more. In alternative embodiments, the carrier may be present in an amount that is less than this range. The structurant (such as hydrogenated castor oil) level may range from about 0 to about 15%, 20%, 30%, 40%, or more. The wax level may range from about 0 to about 3%, 5%, or more. All percentages stated herein are by weight of the composition, unless stated otherwise. The amounts of the components are typically selected so that they total 100%. However, it is also possible for other components to be added to the composition, in which case the weights of the components such as the carrier, volatile material(s), structurant, and wax may total less than 100% of the composition.

The structure (or matrix) comprising the composition can be thermally triggered or otherwise energized to emit the volatile material(s). Such a structure can undergo a transition between a variety of different states depending on the temperature to which the structure is heated. For instance, in some embodiments, the composition can exist in any of the following phases: solid, gel, liquid, and mixtures thereof. Each phase of the composition can provide different volatilization characteristics. In the case of scented materials, this can include different volatilization rates, intensities, scent characters, emission profiles, etc. In some embodiments, the change in state of the composition is reversible in that it can change back to, or toward, more solid states. In some embodiments, it may be possible to vary the form or state of the composition from solid-like to gel-like by controlling the proportions of the components of the composition. For example, the composition will become less solid-like and more gel-like with the addition of additional structurant, such as hydrogenated castor oil. The reversible liquefication/gellation/solidification of the structure can be used to regulate or control the release of the volatile material. In most compositions, in the case of fragrance compositions, at lower temperatures, the more highly volatile perfume components (the “top notes”) will volatilize first. In the case of certain embodiments of the compositions described herein, if the composition is heated above its melting point (until it becomes a liquid), the perception of the volatile composition will be more true to the desired essence of the character, scent, flavor, etc. of the volatile material since all of the components of the material will be emitted at the same intensity at the desired temperature and time from the highly volatile perfume components (the “top notes”) to the less volatile (“bottom notes”). Thus, in certain embodiments, there is minimum partitioning of the volatile material composition and consistency of character/concentration over time. In the case of the examples set out herein, the melting point of the matrix is about 52° C. When energy is no longer applied, the structure goes back to a wax-like solid state or pliable solid which reduces the tendency of the volatile material to escape.

In certain embodiments, it is desirable for the composition to be heated to a temperature that is in excess of the melting point of the carrier. The addition of perfume ingredients will typically lower the melting temperature of the composition. As perfume ingredients are volatilized, the melting temperature of the remaining portion of the composition will increase. If the composition is always heated to a melting temperature above that of the carrier, then this will always provide sufficient energy to the composition in order to emit the volatile components therefrom.

The composition may provide certain advantages. It should be understood in this regard, however, that the composition need not provide any of these advantages unless specified in the appended claims. In some embodiments in the case of fragrance compositions, the composition can deliver a longer lasting aroma. For example, certain gels which have been previously used to contain volatile materials will release the more volatile perfume components even without being heated, or otherwise energized. This will reduce the longevity of such compositions, and will effect the character of the perfume that is emitted when the composition is heated. In some embodiments, the composition can retain the volatile material(s) better than some other compositions during periods when the volatile material(s) are not intended to be emitted. In some embodiments, the composition can be more compatible with the material of the container in which is placed (which may be referred to as “supporting material”). Often perfume oils are not compatible with plastics. However, when perfume oils are incorporated into the composition described herein, the composition may be more compatible with plastic materials. Without wishing to be bound to any particular theory, it is believed that the volatile material-containing composition described herein will have a greater surface tension than that of the perfume oil, to reduce or eliminate migration of the perfume oil from the composition, a phenomenon known as wicking. In some embodiments, the composition will have a surface tension of higher than 20 dyne/cm and lower than 25 dyne/cm. In some embodiments, the composition will have good stability at elevated temperatures (e.g., up to about 120° F., or 50° C.) and/or high humidity (e.g., up to; or greater than or equal to about 80% RH), even at high volatile material concentrations. That is, the composition will not change shape or physical state under such conditions. In certain embodiments, the composition provides a structure that will not change its physical state (e.g., become more liquid) even when it absorbs water, such as humidity.

The composition may, in some embodiments, also be advantageous in that it may contain relatively high levels of volatile material (e.g., from about 25% to about 75% by weight of the composition). The composition can also incorporate a large number, range, spectrum (or portfolio) of different volatile materials. This is possible due to the ability to alter/adjust the polarity of the carrier to match the polarity of the volatile material by modifying the level of the structurant (e.g., hydrogenated castor oil). For example, in the case of the compositions described herein, the polarity of the volatile material(s) can be in the range of from about 2 to about 5 Debyes, yet the compositions may still be stable under a wide range of storage conditions. This allows combinations of perfumes that are typically not compatible to be incorporated into compositions (for example, vanilla, coffee, cinnamon, which are very polar, can be combined with fruits (e.g., lemon), or other types of perfume ingredients that are at the other end of the polarity spectrum. In addition, the structure of the composition that incorporates the volatile material(s) may be reversible (that is, it can be converted from a more solid state (e.g, a pliable solid) to a more liquid state, and then back to a more solid state). This may provide the composition with handling, storing, and processability benefits. The term reversible is used with respect to a change in the physical state of the composition and not to the ability to return to its initial condition. It should be understood that the amount of volatile components released or lost during use is an irreversible process.

In a preferred embodiment, the volatile material of the present invention contains at least a first component and a second component. More preferably, it contains three or more components. At least two of these components preferably have different evaporation rates. Preferably, the volatile material contains at least about 10% by weight of the first component. More preferably, the volatile material contains at least about 20% by weight of the first component. Preferably, the volatile material contains at least about 10% by weight of the second component. More preferably, the volatile material contains at least about 20% by weight of the second component. Preferably, the first component has a boiling point of about 250° C. or less and a ClogP of about 3 or less. Also, the second component preferably has a boiling point of about 250° C. or less and a ClogP of about 3 or more.

In another preferred embodiment, the volatile material comprises at least about 5% by weight of ingredients having a boiling point of greater than or equal to about 250 ° C. and a Clog P value less than or equal to about 3. More preferably, it comprises at least about 10% by weight of these ingredients.

One embodiment of the present invention provides a method releasing a volatile material into the atmosphere while providing a consistent odor profile of the volatile material. The method comprises providing a volatile material-containing composition comprising a carrier; at least one volatile material that is miscible in the carrier, where the volatile material has at least a first component and a second component, where the first component and the second component have different evaporation rates; and at least one polymer having a Hydrophobicity Index greater than about 1.0 and lower than about 3.0 that is miscible in the carrier. The composition has a melting temperature that is lower than the melting temperature of the carrier. The composition has a first state when energy is not applied to the composition, and a second energized state when energy is applied to the composition. The volatile material-containing composition is heated to a temperature above that of the melting temperature of the carrier, resulting in a portion of the volatile material evaporating upon heating. Preferably, when heat is no longer applied to the volatile material-containing composition, the composition returns to the first state.

### EXAMPLES

Table 1 provides some non-limiting examples of scented compositions that can be made according to the description herein.

TABLE 1

| | | Hydrogenated | Wax | Poly(methyl | | |
|---|---|---|---|---|---|---|
| Perfume % | PEG % | Caster Oil % | % | methacrylate) | Polybutadiene | Polystyrene |
| 25 | 70 | 0 | 0 | 5% | | |
| 45 | 45 | 5 | 0 | | 5% | |
| 40 | 30 | 25 | 0 | | | 5% |
| 35 | 40 | 20 | 0 | 2.5% | 2.5% | |
| 40 | 40 | 10 | 2 | | 7% | 1% |
| 42 | 40 | 5 | 3 | 10% | | |
| 25 | 0 | 74 | 0 | | 1% | |

The disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While particular embodiments of the subject invention have been described, it will be obvious to those skilled in the art that various changes and modifications of the subject invention can be made without departing from the spirit and scope of the invention. In addition, while the present invention has been described in connection with certain specific embodiments thereof, it is to be understood that this is by way of illustration and not by way of limitation and the scope of the invention is defined by the appended claims which should be construed as broadly as the prior art will permit.

1. A volatile material-containing composition comprising:
a carrier;
at least one volatile material having at least a first component and a second component, wherein said first component and said second component have different evaporation rates; and
at least one polymer having a Hydrophobicity Index greater than about 1.0 and lower than about 3.0;
wherein said composition has a first non energized and a second energized state, wherein the volatile material is emitted at a first level from said volatile material-containing composition in said first state and at a second higher level in said second state; wherein said volatile material-containing composition returns to said first state when energy is no longer applied, and wherein said composition releases less than 10 mg/hour of said volatile material at 25° C. and 50% relative humidity (RH).

2. A volatile material-containing composition according to claim 1 wherein said carrier has a Hydrophobicity Index less than about 1.0 and said carrier does not interfere with the release profile of said at least one volatile material.

3. A volatile material-containing composition according to claim 1 wherein said at least one volatile material is selected from the group consisting of perfume oils, flavors, pesticides, repellants, and mixtures thereof.

4. The volatile material-containing composition of claim 1 wherein said polymer is selected from the group consisting of polystyrene, bimodal polystyrene, polybutadiene, poly(methyl methacrylate), polyurethane, blends of polyurethane and rosin plasticizer, and mixtures thereof.

5. The volatile material-containing composition of claim 1 wherein said polymer is selected from the group consisting of poly(methyl methacrylate), polybutadiene, and mixtures thereof.

6. The volatile material-containing composition of claim 1 wherein said volatile material comprises:
(a) at least about 10% by weight of a first component, wherein said first component has a boiling point of about 250° C. or less and a ClogP of about 3 or less; and
(b) at least about 10% by weight of a second component, wherein said second component has a boiling point of about 250° C. or less and a ClogP of about 3 or more.

7. The volatile material-containing composition of claim 6 wherein said volatile material comprises:
(a) at least about 20% by weight of said first component; and
(b) at least about 20% by weight of said second component.

8. The volatile material-containing composition of claim 1 wherein said volatile material comprises at least about 10% by weight of volatile ingredients having a boiling point of less than or equal to about 250 ° C. and a Clog P value less than or equal to about 3.

9. The volatile material-containing composition of claim 1 wherein said volatile material comprises at least about 10% by weight of volatile ingredients having a boiling point less than or equal to about 250 ° C. and Clog P value greater than or equal to about 3.

10. The volatile material-containing composition of claim 1 wherein said volatile material comprises at least about 5% by weight of ingredients having a boiling point of greater than or equal to about 250 ° C. and a Clog P value less than or equal to about 3.

11. A volatile material-containing composition according to claim 1 wherein said composition can be thermally triggered at temperatures above 50° C. to emit at least some of the volatile material.

12. The volatile material-containing composition according to claim 1 wherein said composition is melted in said second state.

13. A method of releasing a volatile material into the atmosphere while providing a consistent odor profile of the volatile material comprising:
(a) providing a volatile material-containing composition comprising a carrier; at least one volatile material having at least a first component and a second component, wherein said first component and said second component have different evaporation rates; and at least one polymer having a Hydrophobicity Index greater than about 1.0 and lower than about 2.0; and
(b) heating said volatile material-containing composition to a temperature above the melting temperature of said carrier, wherein a portion of said volatile material is evaporated upon heating;
wherein said at least one volatile material has a melting temperature that is lower than the melting temperature of said carrier, wherein said composition has a first state when energy is not applied to said composition, and a second energized state when energy is applied to said composition.

14. The method of claim 13 wherein when heat is no longer applied to said volatile material-containing composition, said composition returns to said first state.

15. A volatile material-containing composition comprising:
(a) from about 25% to about 75%, by weight, of polyethylene glycol;
(b) from about 10% to about 75%, by weight, of at least one volatile material; and
(c) from about 0.2% to about 3.0%, by weight, of a polymer;
wherein said composition is in the form of a pliable solid at room temperature.

16. The volatile material-containing composition of claim 15 wherein said polymer is selected from the group consisting of polystyrene, bimodal polystyrene, polybutadiene, poly(methyl methacrylate), polyurethane, blends of polyurethane and rosin plasticizer, and mixtures thereof.

17. The volatile material-containing composition of claim 15 wherein said polymer is selected from the group consisting of poly(methyl methacrylate), polybutadiene, and mixtures thereof.

18. A volatile material-containing composition comprising:
(a) a carrier with a Hydrophobicity Index less than about 1.0; and
(b) at least one volatile material;
wherein said carrier releases the volatile material without affecting said volatile material's release profile; and further, wherein said composition has a first non energized and a second energized state, wherein the volatile material is emitted at a first level from said volatile material-containing composition in said first state and at a second higher level in said second state; wherein said volatile material-containing composition returns to said first state when energy is no longer applied, and wherein said composition releases less than 10 mg/hour of said volatile material at 25° C. and 50% relative humidity (RH).

19. A volatile material-containing composition according to claim 18 wherein said at least one volatile material is selected from the group consisting of perfume oils, flavors, pesticides, repellants, and mixtures thereof.

20. A volatile material-containing composition according to claim 18 wherein said carrier is polyethylene glycol and said at least one volatile material comprises perfume oils.
